(19) United States
(12) Patent Application Publication
(10) Pub. No.: US 2026/0034344 A1
(43) Pub. Date: Feb. 5, 2026

(54) CIRCULATION SUPPORT DEVICES, SYSTEMS, AND METHODS

(71) Applicant: BOSTON SCIENTIFIC SCIMED, INC., Maple Grove, MN (US)

(72) Inventors: Darrin Dale Beekman, Osseo, MN (US); Corey N. Meyer, Zimmerman, MN (US)

(73) Assignee: BOSTON SCIENTIFIC SCIMED, INC., Maple Grove, MN (US)

(21) Appl. No.: 19/285,030

(22) Filed: Jul. 30, 2025

Related U.S. Application Data
(60) Provisional application No. 63/678,176, filed on Aug. 1, 2024.

(51) Int. Cl. A61M 60/216; A61M 60/17; A61M 60/416; A61M 60/81

(52) U.S. Cl. CPC A61M 60/216; A61M 60/17; A61M 60/416; A61M 60/81; A61M 2205/0266; A61M 2205/103; A61M 2210/125

(57) ABSTRACT

A mechanical circulatory support system may include a blood pump and a cannula extending distally from the blood pump. The blood pump may include a motor and an impeller assembly in communication with the motor. The impeller assembly may be configured to pump blood from a left ventricle of a heart to an aorta in response to actuation of the motor. The cannula may include a first bend configured to be located proximal of an aortic valve of the patient when the impeller assembly is pumping blood from the left ventricle to the aorta and a second bend configured to be distal of the aortic valve when the impeller assembly is pumping blood from the left ventricle to the aorta.

### CROSS REFERENCE TO RELATED APPLICATIONS

The present application claims the benefit of U.S. Provisional Patent Application Ser. No. 63/678,176, filed Aug. 1, 2024, the disclosure of which is incorporated herein by reference.

### TECHNICAL FIELD

The present disclosure pertains to mechanical circulatory support devices and systems. More specifically, the present disclosure relates to devices, systems, and methods of and/or for positioning mechanical circulatory support devices and systems.

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, cardiac assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

A first example may include a mechanical circulatory support system comprising a motor, an impeller assembly in communication with the motor and the impeller assembly is configured to pump blood from a left ventricle of a heart of a patient to an aorta of the patient in response to actuation of the motor, a cannula extending distally from the impeller assembly, wherein the cannula may include a first bend configured to be located concentrically within or proximal of an aortic valve of the patient when the impeller assembly is pumping blood from the left ventricle to the aorta and a second bend configured to be located distal of the aortic valve when the impeller assembly is pumping blood from the left ventricle to the aorta.

Alternatively or additionally to any of the examples above, the cannula may comprise one or more inflow openings and the second bend may be configured to orient the one or more inflow openings through a center of a left ventricular outflow tract (LVOT) of the left ventricle when the impeller assembly is pumping blood from the left ventricle to the aorta.

Alternatively or additionally to any of the examples above, the first bend may be configured to position the cannula within a central portion of a cross-section extending through the aortic valve when the impeller assembly is pumping blood from the left ventricle to the aorta.

Alternatively or additionally to any of the examples above, the first bend may define a first angle extending in a first plane and the second bend may define a second angle extending in a second plane different than the first plane.

Alternatively or additionally to any of the examples above, an angle between the first plane and the second plane may be in a range of 60 degrees to 140 degrees.

Alternatively or additionally to any of the examples above, the angle between the first plane and the second plane may be 105 degrees.

Alternatively or additionally to any of the examples above, the first angle may be in a range of 100 degrees to 140 degrees and the second angle may be in a range of 120 degrees to 160 degrees.

Alternatively or additionally to any of the examples above, the cannula at the second bend may be formed from a polymer.

Alternatively or additionally to any of the examples above, the cannula at the second bend may be formed from a shape set nickel-titanium alloy.

Alternatively or additionally to any of the examples above, the shape set nickel-titanium alloy may be a shape memory nickel-titanium alloy with a transition temperature in a range of twenty degrees Celsius to forty degrees Celsius.

Alternatively or additionally to any of the examples above, the impeller assembly may comprise an impeller and an impeller housing, and the impeller may rotate in response to actuation of the motor to draw blood from the left ventricle into the cannula and through the impeller housing into the aorta.

In another example, a mechanical circulatory support system may include a blood pump configured to pump blood from a left ventricle of a heart of a patient to an aorta of the patient and a cannula extending distally from the blood pump, wherein the cannula may comprise a first bend having a first angle configured to direct the cannula through an aortic valve of the patient when the blood pump is pumping blood from the left ventricle to the aorta and a second bend having a second angle configured to direct a distal end of the cannula toward an apex of the left ventricle.

Alternatively or additionally to any of the examples above, the first angle may extend in a first plane and the second angle may extend in a second plane different than the first plane.

Alternatively or additionally to any of the examples above, the first bend may be configured to be located proximal of the aortic valve when the blood pump is pumping blood from the left ventricle to the aorta and the second bend may be configured to be located distal of the aortic valve when the blood pump is pumping blood from the left ventricle to the aorta.

Alternatively or additionally to any of the examples above, the first angle may extend in a first plane, the second angle may extend in a second plane, and an angle between the first plane and the second plane may be in a range of 60 degrees to 140 degrees.

Alternatively or additionally to any of the examples above, the first angle may be in a range of 100 degrees to 140 degrees and the second angle may be in a range of 120 degrees to 160 degrees.

In another example, a method may include inserting a mechanical circulatory support system into a vasculature of a patient, the mechanical circulatory support system comprising a blood pump and a cannula extending distally from the blood pump, delivering the mechanical circulatory support system through the vasculature to a heart of the patient, and orienting a distal end of the cannula toward an apex of a left ventricle of the heart.

Alternatively or additionally to any of the examples above, orienting the distal end of the cannula toward the apex of the left ventricle may comprise positioning a bend in the cannula distal of an aortic valve of the patient.

Alternatively or additionally to any of the examples above, orienting the distal end of the cannula toward the apex of the left ventricle may comprise positioning a first bend in the cannula proximal of an aortic valve of the patient and positioning a second bend in the cannula distal of the aortic valve of the patient.

Alternatively or additionally to any of the examples above, the method may further include orienting a proximal portion of the cannula in a central portion of an aortic valve of the patient.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative circulatory support system;

FIG. 2 is a schematic partial cross-section view of anatomy and a schematic side view of an illustrative mechanical circulatory support (MCS) system within the anatomy;

FIG. 3 is a schematic cross-section view of a portion of an introducer sheath within a blood vessel and a portion of an illustrative MCS system in the introducer sheath and the blood vessel;

FIG. 4 is a schematic cross-section view of anatomy and a schematic side view of an illustrative MCS system within the anatomy;

FIG. 5 is a schematic perspective view of a portion of an illustrative MCS system;

FIGS. 6A and 6B are schematic perspective views of a portion of an illustrative cannula of the MCS system depicted in FIG. 5; and

FIG. 7 is a schematic cross-section view of anatomy and a schematic perspective view of the illustrative MCS system depicted in FIG. 5 within the anatomy.

While the disclosure is amenable to various modifications and alternative forms, specifics thereof have been shown by way of example in the drawings and will be described in detail. The intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support systems or devices may support a patient during percutaneous coronary interventions and/or other procedures.

Example cardiac circulatory support systems or devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

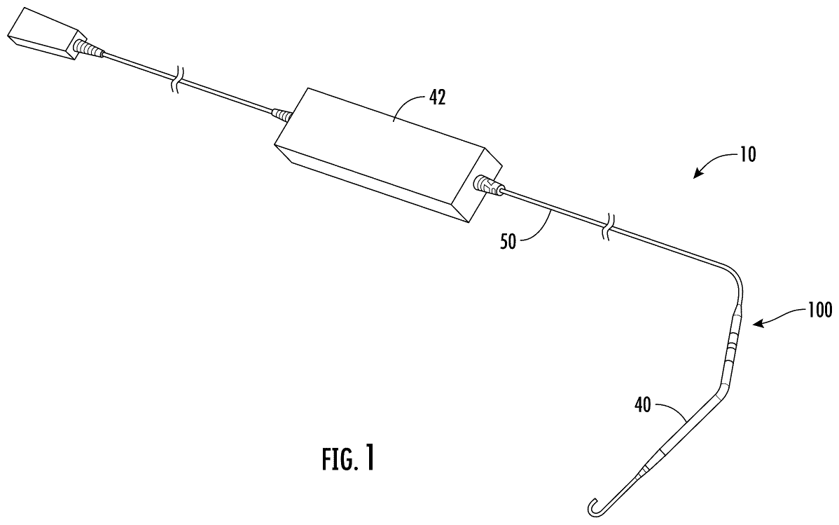

FIG. 1 depicts a schematic view of an illustrative circulatory support system 10 (e.g., a mechanical circulatory support (MCS) system). The system 10 may include a percutaneous support device (e.g., a PVAD, such as a blood pump 100), a cannula 40, an elongate tube 50, and an introducer sheath (not shown). In some examples, the system 10 may include a guidewire, but this is not required. The introducer sheath, when included, may facilitate percutaneous delivery of the blood pump 100 and the cannula 40 to a target location within a patient (e.g., a target location within a heart of a patient). When positioned at a target site in a heart of a patient, the blood pump 100 may be configured to pump blood from a ventricle of the heart to vasculature of the patient.

The cannula 40 may be a component of the blood pump 100 and/or a separate component coupled with the blood pump 100. In some examples, although the cannula 40 may be discussed as extending from the blood pump 100, the cannula 40 may be part of the blood pump 100 (e.g., formed from a component of the blood pump, such as an impeller housing and/or other suitable component), but other suitable configurations are contemplated.

The system 10 may further include a proximal housing 42 coupled with (e.g., connected to) the elongate tube 50 (e.g., a catheter and/or other suitable elongate tube), where the elongate tube 50 may be coupled with the blood pump 100 (e.g., a distal end of the elongate tube 50 may be coupled with a proximal end of the blood pump 100) and one or more wires and/or shafts of or coupled with the blood pump 100 may extend proximally from the blood pump 100 through the tube 50. In some examples, at least the proximal housing 42, the elongate tube 50, and/or other components of the system 10 may be configured to facilitate delivering the blood pump 100 and the cannula 40 to the target location or site within the patient. The elongate tube 50 and the proximal housing 42 may be configured to be positioned at least partially exterior of the patient when the blood pump 100 and the cannula 40 are at the target location. In some examples, the guidewire, when included, may also be used to facilitate the delivery of the blood pump 100 and/or the cannula 40.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and/or during delivery of the blood pump 100 and the cannula 40 to a target site.

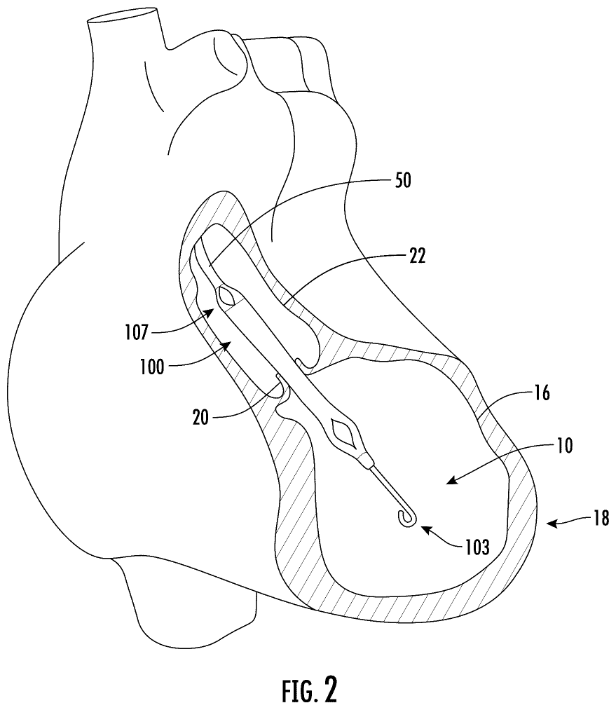

FIG. 2 depicts a schematic view of an illustrative positioning of the blood pump 100 (e.g., a percutaneous circulatory support device, such as a PVAD, etc.) in anatomy of a patient (e.g., in a left ventricle 16 in a heart 18). As depicted in FIG. 2, the blood pump 100 may be positioned with a distal end 103 thereof located in a left ventricle 16 of the heart 18 and a proximal end 107 of the blood pump 100 in an aorta 22, such that the blood pump 100 (e.g., which may or may not include the cannula 40) extends across an aortic valve 20 between the left ventricle 16 and the aorta 22. With the blood pump 100 extending from the left ventricle 16 to the aorta 22 of the patient, the blood pump 100 may be configured to pump blood from the left ventricle 16 into the aorta 22 (e.g., into the ascending aorta) to assist or support blood flow circulation. Other suitable positions of the blood pump 100 relative to the anatomy of the patient are contemplated and include, but are not limited to, the distal end 103 of the blood pump 100 being positioned in or in fluid communication with a right ventricle of the heart 18 with the proximal end 107 of the blood pump 100 being positioned in a pulmonary artery.

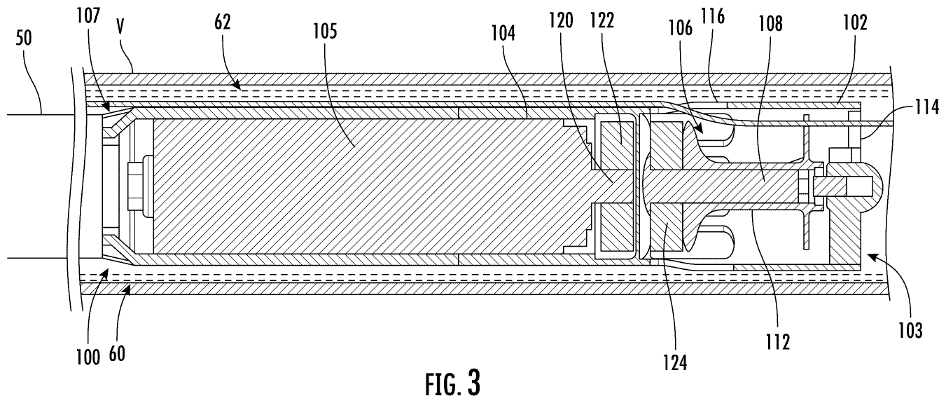

FIG. 3 depicts a schematic cross-section view of a body portion 62 of an introducer sheath 60 extending into a blood vessel V with the blood pump 100 inserted into the introducer sheath 60. The blood pump 100 may include and/or may be coupled with the elongate tube 50 extending proximally from the proximal end 107 of the blood pump 100 to a location outside of the blood vessel V and the introducer sheath 60. When positioning the blood pump 100 within the patient, the blood pump 100 may be advanced through the blood vessel V and positioned in or at a target location, such as a target cardiac location (e.g., in the aorta 22, across the aortic valve 20, and/or into the left ventricle 16), via the introducer sheath 60. While the introducer sheath 60 is illustrated in FIG. 3 with the use of the blood pump 100, various other medical devices may be used in conjunction with the introducer sheath 60.

The blood pump 100 may include one or more components. In some examples, the blood pump 100 may include an impeller housing 102 and a motor housing 104. The impeller housing 102 and the motor housing 104 may be integrally or monolithically constructed. Alternatively, the impeller housing 102 and the motor housing 104 may be separate components. The impeller housing 102 may carry an impeller assembly 106 therein. The impeller assembly 106 may include an impeller shaft 108 and an impeller 112 that rotates relative to the impeller housing 102 to drive or pump blood from the left ventricle 16 of the heart 18 of the patient (e.g., via the cannula 40 of or in fluid communication with the blood pump 100) to the aorta 22 or portion of the anatomy in communication with the aorta (e.g., pump blood through the blood pump 100 to an ascending aorta). In some examples, the impeller shaft 108 and the impeller 112 may be integrally formed, whereas, in other examples the impeller shaft 108 and the impeller 112 may be separate components.

Rotation of the impeller 112 may cause blood to flow from a blood inlet 114 formed on the impeller housing 102 (e.g., where the blood may enter the blood inlet 114 directly from the left ventricle and/or via the cannula 40), through the impeller housing 102, and out of a blood outlet 116 formed on the impeller housing 102. As shown in FIG. 3, the blood inlet 114 may be formed on an end portion of the impeller housing 102 and the blood outlet 116 may be formed on a side portion of the impeller housing 102. In other examples, the blood inlet 114 and/or the blood outlet 116 may be formed on or at other portions of the impeller housing 102 or other components of the system 10.

The impeller housing 102 of the impeller assembly 106 may be coupled to the cannula 40 and the cannula 40 may extend distally from the impeller housing 102 (e.g., extending distally from the distal end 103 of the blood pump 100), and the cannula 40 may receive blood from the left ventricle and deliver the blood to the blood inlet 114. Alternatively or additionally, the impeller housing 102 may be further elongated than depicted in FIG. 3 and define all of or at least part of the cannula 40 extending into the left ventricle of the patient. Other suitable configurations of the impeller housing 102 and/or the cannula 40 are contemplated.

The motor housing 104 may carry a motor 105, and the motor 105 may be configured to rotatably drive the impeller 112 relative to the impeller housing 102 when the motor 105 is actuated. In some examples, the motor 105 may rotate a drive shaft 120 coupled with a driving magnet 122. Rotation of the driving magnet 122 may cause rotation of a driven magnet 124 that may be coupled with and/or may be part of the impeller assembly 106. More specifically, in configurations incorporating the impeller shaft 108, the impeller shaft 108 and the impeller 112 may be configured to rotate with the driven magnet 124. In other configurations, the motor 105 and/or the motor shaft 120 may be coupled to the impeller assembly 106 directly and/or via other suitable components.

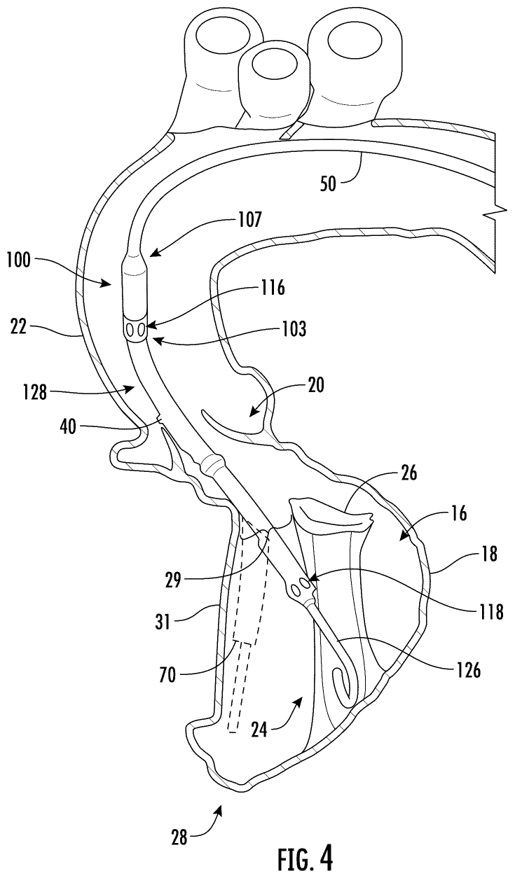

FIG. 4 depicts a schematic cross-section view of anatomy of a patient with a schematic side view of a portion of an illustrative configuration of the system 10 extending within the anatomy. The anatomy schematically depicted in FIG. 4 includes, among other anatomy, the aorta 22, the aortic valve 20, the left ventricle 16, the mitral valve 26, and a mitral valve apparatus 24.

When the system 10 is positioned within the anatomy of the patient, for example as depicted in FIG. 4, the blood pump 100 may be positioned at the aorta 22, the cannula 40 may extend distally from the distal end 103 of the blood pump 100, across the aortic valve 20, and into the left ventricle 16 of the heart 18 (e.g., schematically depicted with chambers other than the left ventricle 16 omitted), and the elongate tube 50 may extend proximally from a proximal end 107 of the blood pump 100 through the aorta 22 and eventually out of patient (e.g., via the femoral artery and/or other suitable vessel access site). When so positioned, inflow openings 118 of the cannula 40 may be positioned within the left ventricle 16 and the blood outlet 116 may be located in the aorta 22.

An atraumatic distal tip 126 may form a distal end of the cannula 40 and/or may extend distally from the distal end of the cannula 40, but the atraumatic distal tip 126 may be omitted. When included, the atraumatic distal tip 126 may be bent to form a rounded distal end, but other suitable configurations are contemplated.

The blood pump 100 and/or the cannula 40 may be configured to facilitate positioning the blood pump 100 and/or the cannula 40 in a desired manner within the heart 18 and aorta 22 of the patient. For example, the blood pump 100 and/or the cannula 40 may be configured to center the cannula 40 within the aortic valve 20, position the blood outlet 116 in the aorta 22, position the inflow openings 118 within the left ventricle 16, and position the inflow openings 118 at a location within the left ventricle that does not interfere with the mitral valve apparatus 24 configured to facilitate operation of a mitral valve 26 (e.g., at a location such that inflow of blood into the inflow openings 118 does not suction the mitral valve apparatus 24 and/or otherwise interfere with operation of the mitral valve 26).

To facilitate positioning the blood pump 100 and/or the cannula 40 within the heart 18 or other suitable target location in a desired manner, the blood pump 100 and/or cannula 40 may include one or more bends. For example, as depicted in FIG. 4, the cannula 40 may include a single bend 128. In some examples, the single bend 128 may be configured to facilitate crossing the aortic valve 20 with the cannula 40 and mitigate damage to the aortic valve 20 as the cannula 40 is positioned within the left ventricle 16. Further, the single bend 128 of the cannula 40 may be configured to be located proximal of the aortic valve 20 when the blood pump 100 and the cannula 40 are at a target location within the heart 18 such that the single bend 128 may be configured to center the cannula 40 within the aortic valve 20 to facilitate coaptation of leaflets of the aortic valve 20 with the cannula 40 as the leaflets adjust from an opened position to a closed position.

Although the single bend 128 of the cannula 40 may facilitate crossing the aortic valve 20 and positioning the cannula 40 within the aortic valve 20 in a desired manner, the single bend 128 may have an unintended consequence of pointing and/or positioning the cannula 40 (e.g., the inflow openings 118 of the cannula 40) at and/or in the mitral valve apparatus 24 and/or the mitral valve 26. Such positioning of the cannula 40 within the left ventricle 16 may cause damage to and/or interfere with the mitral valve apparatus 24 and/or the mitral valve 26 due to engagement and/or suctioning of the mitral valve apparatus 24 and may reduce blood flow through the cannula 40 and the blood pump 100 relative to when inflow openings 118 of the cannula 40 are positioned in more suitable manners within the left ventricle 16.

Instead of positioning the inflow openings 118 of the cannula 40 in or at the mitral valve apparatus 24, it is desired to position the cannula 40 such that the distal end thereof and/or the inflow openings 118 are located in or through a center or central portion of a left ventricular outflow tract (LVOT) 29 in the left ventricle 16 typically with the distal tip of the cannula 40 directed toward the apex 28 of the left ventricle 16 (e.g., as depicted in broken or phantom lines 70 in FIG. 4). The LVOT 29 may be a tract, channel, or region extending between a ventricular septum 31 and an anterior cusp of the mitral valve 26.

To facilitate positioning the cannula 40 in a central portion of the aortic valve 20 and/or facilitate positioning and/or orienting the distal end of the cannula 40 toward the apex 28 and/or the inflow openings 118 in or through the center of the LVOT 29 and away from the mitral valve apparatus 24, the cannula 40 may include a first bend and a second bend. In some examples, the first bend of the cannula 40 may be configured to be located proximal of the aortic valve 20 and to position the cannula 40 in a central portion of the aortic valve 20 (e.g., similar to the single bend 128) and the second bend may be located distal of the aortic valve 20 to position cannula 40 with the distal end thereof and the inflow openings 118 directed at the apex 28 of the left ventricle 16 when the blood pump 100 and the cannula 40 are located at the target location within the heart 18 of the patient and/or when the impeller assembly 106 is pumping blood from the left ventricle 16 to the aorta 22 or other suitable portion of anatomy.

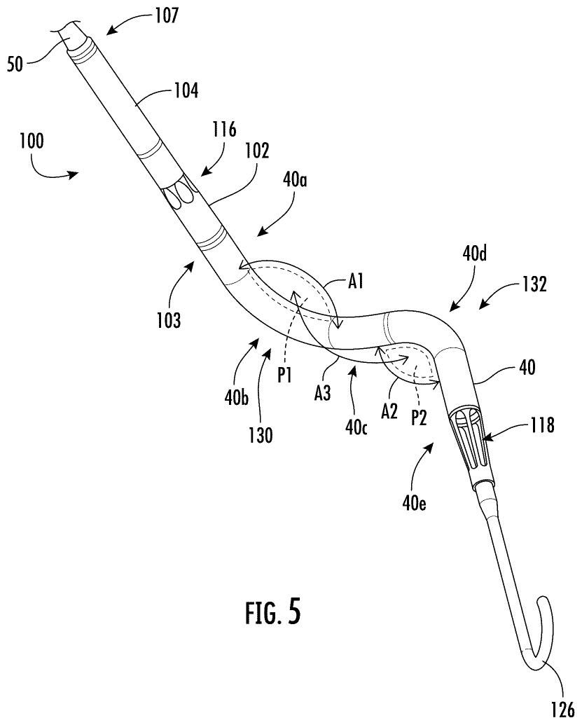

FIG. 5 depicts a schematic perspective view of a portion of an illustrative configuration of the system 10 with the cannula 40 having a first bend 130 and a second bend 132. Although the first bend 130 and the second bend 132 are depicted in FIG. 5 and described as being formed in the cannula 40, the first bend 130 and the second bend 132 may be formed in other suitable components of the system 10 including, but not limited to, the blood pump 100, the impeller housing 102, the motor housing 104, and/or other suitable components.

The cannula 40 may have one or more segments or portions defining features of the cannula 40. In one example, when the cannula 40 is coupled with the distal end 103 of the blood pump 100 (e.g., coupled with a distal end of the impeller housing 102), a first portion 40a of or at a proximal end of the cannula 40 may couple with the distal end 103 of the blood pump 100, a second portion 40b of the cannula 40 may extend distally from the first portion 40a and define the first bend 130, a third portion 40c of the cannula 40 may extend distally from the second portion 40b and may be configured to extend across the aortic valve 20, a fourth portion 40d may extend distally from the third portion 40c and define the second bend 132, and a fifth portion 40c may extend distally from the fourth portion 40d. In some examples, the fifth portion 40c of the cannula 40 may include or form the inflow openings 118 or the inflow openings 118 may be separate from the fifth portion 40e (e.g., the inflow openings 118 may be part of a sixth portion of the cannula 40 and/or may be separate from the cannula 40). Other suitable configurations of the cannula 40 with additional and/or alternative segments or portions are contemplated.

The segments or portions of the cannula 40 may have any suitable axial lengths. In some examples, the first portion 40a of the cannula 40 may have a length in a range of 0 millimeters (mm) to 20 mm, the second portion 40b of the cannula 40 may have a length in a range of 10 mm to 30 mm, the third portion 40c of the cannula 40 may have a length in a range of 0 mm to 20 mm, the fourth portion 40d of the cannula 40 may have a length in a range of 10 mm to 30 mm, and the fifth portion 40e of the cannula 40 may have a length in a range of 0 mm to 20 mm. The first portion 40a, the third portion 40c, and the fifth portion 40e may have lengths of 0 mm when such portions are omitted and the curves defined by the second portion 40b and the fourth portion 40d run together without an extension portion and/or an intervening portion. In one example configuration, the first portion 40a of the cannula 40 may have a length of 7.62 mm, the second portion 40b of the cannula 40 may have a length of 14.63 mm, the third portion 40c of the cannula 40 may have a length of 10.07 mm, the fourth portion 40d of the cannula 40 may have a length of 20.80 mm, and the fifth portion 40e of the cannula 40 may have a length of 7.62 mm. Other suitable lengths of the portions of the cannula 40 are contemplated.

The first bend 130 and the second bend 132 may have any suitable configurations. In some examples, the first bend 130 and/or the second bend 132 may be configured individually and/or relative to one another such that the bends 130, 132 are configured to facilitate centering the cannula 40 in the aortic valve 20 and orienting the inflow openings 118 toward or through the center or central portion of the LVOT 29 in the left ventricle 16 and the distal end or distal tip of the cannula 40 toward the apex 28 of the left ventricle 16 in a variety of patient anatomy configurations (e.g., mid-deep configuration of the left ventricle 16, boot-shape configuration of the left ventricle 16, etc.

The first bend 130 may have any suitable angle A1. In some examples, the angle A1 of the first bend 130 may be in a first plane P1 (e.g., including or parallel to one or more axes of the first portion 40a, the second portion 40b, and the third portion 40c of the cannula 40) and may be configured to position the cannula 40 or other suitable component of the system 10 in a central portion of a cross-section of an opening through the aortic valve 20 (e.g., where the opening may be entirely or at least partially defined leaflets of the aortic valve 20 when the leaflets are in an opened position), where the cross-section may be taken at an axial location of the opening at which leaflets of the aortic valve 20 coapt with or are intended to coapt one another when in a closed position. In some examples, the angle A1 of the first bend 130 may be within a range of 100 degrees to 140 degrees. In one example, the angle A1 of the first bend 130 may be or may be approximately 105 degrees, but other suitable angles are contemplated.

The central portion of the cross-section of the opening through the aortic valve 20 may be or may be about a central axis of the opening (e.g., an axis centrally located in the opening) and/or may be or may be about a shape centered on the central axis of the opening, where a perimeter of the shape is spaced radially inward from an outer perimeter of the opening. For example, the central portion of the cross-section of the opening may have an area defined by a circle, a triangle, and/or other suitable shape centered on the central axis with a maximum distance from the central axis to a perimeter of the suitable shape (e.g., a radius in the case of a circle) that is less than a shortest distance from the central axis to a wall of the aortic valve 20. In some examples, the central portion may have an area that may be ninety percent, eighty percent, seventy-five percent, two thirds, fifty percent, one third, twenty-five percent, ten percent, or other suitable portion of an area defined by the cross-section of the opening through the aortic valve 20. In one example, the first bend 130 may be configured to position the cannula 40 within a central portion of the aortic valve 20 that defines an area equal to or less than fifty percent of an area defined by the opening through the aortic valve 20. In one example, the first bend 130 may be configured to position the cannula 40 along the central axis of the opening through the aortic valve 20 when the cannula 40 is positioned in the central portion of the aortic valve 20.

The second bend 132 may have any suitable angle A2. In some examples, the angle A2 of the first bend 130 may be in a second plane P2 (e.g., including or parallel to one or more axes of the third portion 40c, the fourth portion 40d, and the fifth portion 44e of the cannula 40) and configured to position and/or orient the distal end of the cannula 40 and/or the inflow openings 118 in the left ventricle 16 such that the cannula 40 and/or the inflow openings 118 are directed toward the center of the LVOT 29 within the left ventricle 16. In some examples, the angle A2 of the second bend 132 may be within a range of 120 degrees to 160 degrees. In one example, the angle A2 of the second bend 132 may be or may be approximately 140 degrees, but other suitable angles are contemplated.

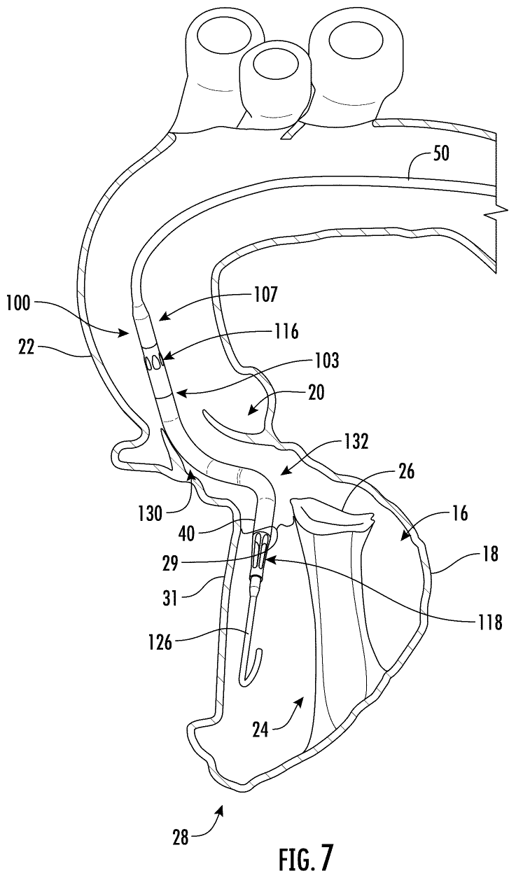

An orientation of the first plane P1 relative to the second plane P2 may be configured to match an orientation of anatomy through which the cannula 40 may extend. For example, the first plane P1 and the second plane P2 may be configured to be in-line with one another, parallel to one another, or intersect and form an angle A3 based on an anatomy of a patient. In some examples, when the first plane P1 and the second plane P2 intersect, the angle A3 may be or may approximate an angle between a plane of the ascending aorta and a plane of the left ventricle 16 to facilitate positioning and/operating the blood pump 100 and/or the cannula 40 at the target location within the patient. In one example, the angle A3 may be or may approximate an angle between the plane created from the ascending aorta and aortic arch (e.g., shown as the aorta 22 depicted in FIGS. 4 and 7) and a plane created from the apex 28 of the left ventricle 16, the center of the aortic valve 20, and the center of the ascending aorta (e.g., a center line of the aorta 22 through a sinotubular junction).

The angle A3 between the first plane P1 and the second plane P2 may be any suitable angle. In some examples, the angle A3 between the first plane P1 and the second plane P2 may be within a range of 60 degrees to 140 degrees. In one example, the angle A3 between the first plane P1 and the second plane P2 may be or may be approximately 105 degrees, but other suitable angles are contemplated.

The cannula 40 (e.g., an entirety of the cannula 40, the cannula 40 at the first bend 130, the cannula 40 at the second bend 132, and/or at other locations along the cannula 40 and/or the blood pump 100) may be formed from any suitable material configured to facilitate delivering the system 10 to the target location in the heart 18 of the patient and forming the first bend 130 and/or second bend 132 when the blood pump 100 and the cannula 40 are positioned at the target location within the aorta 22 and/or the heart 18. The material of the cannula 40 may be selected based on one or more considerations including, but not limited to, kink resistance, lubricity, flexibility, rigidity or stiffness, resilience, shape memory properties, and/or other suitable considerations. Example suitable materials include, but are not limited to, metals, polymers, shape memory materials, shape set materials, nickel-titanium alloys (e.g., NITINOL and/or other suitable nickel-titanium alloys), stainless steel, polytetrafluoroethylene (PTFE), polyamid (e.g., VESTAMID and/or other suitable polyamid material) and/or other suitable materials. In some examples, the cannula 40 and/or portions thereof may be formed from a shape set nickel-titanium alloy. In some examples, the cannula 40 and/or portions thereof may be formed from stainless steel. In some examples, the cannula 40 and/or portions thereof may be formed from a polymer material. In some examples, the cannula 40 and/or portions thereof may be formed from a metal inner tube and polymer outer tube.

The material may be formed in any suitable manner that is configured to form the illustrative configurations of the cannula 40 discussed herein. For example, the material of the cannula 40 may be formed in a tubular manner, formed into a braid, formed into a coil, formed into a hypotube, formed into a slotted hypotube, reflowed, molded, coated, and/or formed in one or more other suitable manners. In some examples, the cannula 40 may be formed from a shape set nickel-titanium alloy hypotube having laser cuts. In some examples, the cannula 40 may be formed from a shape set nickel-titanium alloy hypotube having a coil configuration. In some examples, the cannula 40 may be formed from a shape set polymer. In some examples, the cannula 40 may be formed from a metallic or polymer inner tube having a braid, coil, and/or slotted hypotube configuration and one or more both of a reflowed, molded, or coated polymer outer tube or layer and a reflowed, molded, or coated inner tube or layer. In some examples, the cannula 40 or at least portions at or proximate the first bend 130 and/or the second bend 132 may be formed from a shape memory nickel-titanium alloy hypotube that is heat set to transition from a delivery configuration (e.g., a straight or non-bend configuration) to an operational configuration (e.g., a configuration with the single bend 128, a configuration with the first bend 130 and the second bend 132, etc.) at a transition temperature. When so configured, the transition temperature may be a temperature in a range from room temperature to a body temperature, a temperature in a range from 20 degrees Celsius to 40 degrees Celsius. In one example, the transition temperature may be 37 degrees Celsius, but other suitable transition temperatures are contemplated.

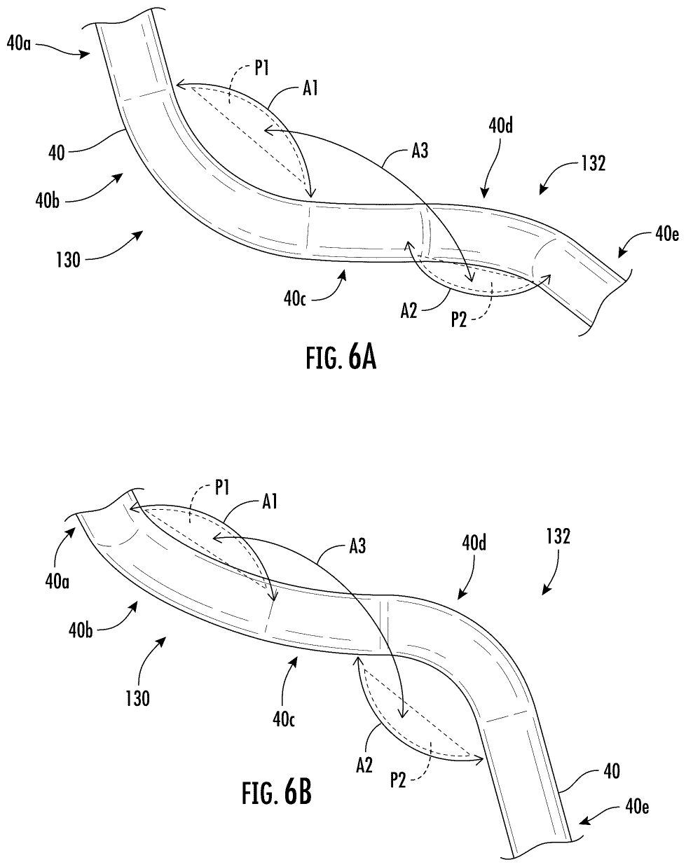

FIGS. 6A and 6B schematically depict a portion of the cannula 40 of the illustrative configuration of the system 10 depicted in FIG. 5 from different perspectives. FIG. 6A schematically depicts the portion of the cannula 40 with the plane P1 of the angle A1 of the first bend 130 parallel with a surface or plane on which the figure is located. FIG. 6B schematically depicts the portion of the cannula 40 with the plane P2 of the angle A2 of the second bend 132 parallel with the surface or plane on which the figure is located. The different views of the portion of the cannula depicted in FIGS. 6A and 6B may demonstrate the angle A3 between planes P1 and P2 or the first portion 40a and the fifth portion 40e of the cannula.

FIG. 7 schematically depicts the blood pump 100 and the cannula 40 of the illustrative configuration of the system 10 depicted in FIG. 5 positioned at a target location within anatomy of the patient. For example, the blood pump 100 is positioned in the aorta 22 proximate the heart 18. The elongate tube 50 may extend proximally from the proximal end 107 of the blood pump 100 through the aorta 22 to a location exterior of the patient. The cannula 40 may extend distally from the distal end 103 of the blood pump 100 and into the left ventricle 16. As depicted in FIG. 7, the first bend 130 may be located proximal of the aortic valve 20 and may facilitate positioning the cannula 40 in a central portion of the aortic valve 20 when the impeller assembly 106 is pumping blood from the left ventricle 16 to the aorta 22 (e.g., the ascending aorta) and/or other suitable portion of anatomy). The second bend 132 may be located distal of the aortic valve 20 and may facilitate positioning the cannula 40 such that the inflow openings 118 are directed and/or oriented toward the center or central portion of the LVOT 29 of the left ventricle 16 and/or the distal end or distal tip of the cannula 40 is directed at and/or oriented toward the apex 28 and away from the mitral valve apparatus 24 when the impeller assembly 106 is pumping blood from the left ventricle 16 to the aorta and/or other suitable portion of anatomy. When the blood pump 100 and the cannula 40 are so positioned, the motor 105 may be actuated to cause rotation of the impeller 112 to draw blood from the left ventricle 16 into the cannula 40 and through the impeller housing 102 into the aorta 22 (e.g., the ascending aorta or other suitable portion of the aorta 22) and/or other suitable anatomy in communication with the aorta 22.

To position the system 10 as depicted in FIG. 7, the system 10 may be inserted into the vasculature of the patient via a vessel access site (e.g., a femoral artery access site and/or other suitable access site). In some examples, the blood pump 100 and the cannula 40 of the system 10 may be inserted into the vasculature in a delivery configuration (e.g., with any bends in the cannula 40 at less severe angles than when the cannula 40 is at a target location and/or in operation at the target location). The system 10, including the blood pump 100 and the cannula 40, may be delivered through the vasculature of the patient (e.g., through the aorta 22 of the patient and/or other suitable vasculature) to the heart 18 of the patient.

When at the heart 18, the cannula 40 of the system 10 may be inserted into the left ventricle 16 and a distal end of the cannula 40 and/or the inflow openings 118 may be oriented toward the apex 28 of the left ventricle 16. To facilitate orienting the distal end or distal tip of the cannula 40 toward the apex 28 of the left ventricle 16 and/or the inflow openings 118 toward the center or central portion of the LVOT 29 of the left ventricle 16, the first bend 130 of the cannula 40 may be positioned or located proximal of the aortic valve 20 and the second bend 132 of the cannula 40 may be positioned or located distal of the aortic valve 20. Further, positioning the first bend 130 of the cannula 40 proximal of the aortic valve 20 may facilitate positioning or orienting a portion of the cannula 40 extending through the aortic valve 20 in a central portion of the aortic valve 20. Positioning or orienting the cannula 40 in the central portion of the aortic valve 20 may facilitate leaflets of the aortic valve 20 coapting around the cannula 40.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A mechanical circulatory support system comprising:
a motor;
an impeller assembly in communication with the motor and the impeller assembly is configured to pump blood from a left ventricle of a heart of a patient to an aorta of the patient in response to actuation of the motor;
a cannula extending distally from the impeller assembly, and
wherein the cannula comprises:
a first bend configured to be located concentrically within or proximal of an aortic valve of the patient when the impeller assembly is pumping blood from the left ventricle to the aorta, and
a second bend configured to be located distal of the aortic valve when the impeller assembly is pumping blood from the left ventricle to the aorta.

2. The system of claim 1, wherein the cannula comprises one or more inflow openings and the second bend is configured to orient the one or more inflow openings toward a central portion of the left ventricular outflow tract (LVOT) of the left ventricle when the impeller assembly is pumping blood from the left ventricle to the aorta.

3. The system of claim 1, wherein the first bend is configured to position the cannula within a central portion of a cross-section extending through the aortic valve when the impeller assembly is pumping blood from the left ventricle to the aorta.

4. The system of claim 1, wherein the first bend defines a first angle extending in a first plane and the second bend defines a second angle extending in a second plane different than the first plane.

5. The system of claim 4, wherein an angle between the first plane and the second plane is in a range of 60 degrees to 140 degrees.

6. The system of claim 5, wherein the angle between the first plane and the second plane is 105 degrees.

7. The system of claim 4, wherein the first angle is in a range of 100 degrees to 140 degrees and the second angle is in a range of 120 degrees to 160 degrees.

8. The system of claim 1, wherein the cannula at the second bend is formed from a polymer.

9. The system of claim 1, wherein the cannula at the second bend is formed from a shape set nickel-titanium alloy.

10. The system of claim 9, wherein the shape set nickel-titanium alloy is a shape memory nickel-titanium alloy with a transition temperature in a range of 20 degrees Celsius to 40 degrees Celsius.

11. The system of claim 1, wherein the impeller assembly comprises an impeller and an impeller housing, and the impeller rotates in response to actuation of the motor to draw blood from the left ventricle into the cannula and through the impeller housing into the aorta.

12. A mechanical circulatory support system comprising:
a blood pump configured to pump blood from a left ventricle of a heart of a patient to an aorta of the patient;
a cannula extending distally from the blood pump, and
wherein the cannula comprises:
a first bend having a first angle configured to direct the cannula through an aortic valve of the patient when the blood pump is pumping blood from the left ventricle to the aorta, and
a second bend having a second angle configured to direct a distal end of the cannula toward an apex of the left ventricle.

13. The system of claim 12, wherein the first angle extends in a first plane and the second angle extends in a second plane different than the first plane.

14. The system of claim 12, wherein:
the first bend is configured to be located proximal of the aortic valve when the blood pump is pumping blood from the left ventricle to the aorta, and
the second bend is configured to be located distal of the aortic valve when the blood pump is pumping blood from the left ventricle to the aorta.

15. The system of claim 12, wherein the first angle extends in a first plane, the second angle extends in a second plane, and an angle between the first plane and the second plane is in a range of 60 degrees to 140 degrees.

16. The system of claim 12, wherein the first angle is in a range of 100 degrees to 140 degrees and the second angle is in a range of 120 degrees to 160 degrees.

17. A method, comprising:
inserting a mechanical circulatory support system into a vasculature of a patient, the mechanical circulatory support system comprising:
a blood pump;
a cannula extending distally from the blood pump;
delivering the mechanical circulatory support system through the vasculature to a heart of the patient; and
orienting a distal end of the cannula toward an apex of a left ventricle of the heart.

18. The method of claim 17, wherein orienting the distal end of the cannula toward the apex of the left ventricle comprises positioning a bend in the cannula distal of an aortic valve of the patient.

19. The method of claim 17, wherein orienting the distal end of the cannula toward the apex of the left ventricle comprises positioning a first bend in the cannula proximal of an aortic valve of the patient and positioning a second bend in the cannula distal of the aortic valve of the patient.

20. The method of claim 17, further comprising:
orienting a proximal portion of the cannula in a central portion of an aortic valve of the patient.
